(19) United States
(12) Patent Application Publication
(10) Pub. No.: US 2013/0032967 A1
(43) Pub. Date: Feb. 7, 2013

(54) COLD ETHYLENE OXIDE STERILIZATION OF A BIODEGRADABLE POLYMERIC STENT

(71) Applicants: Yunbing Wang, Sunnyvale, CA (US); Byron J. Lambert, Temecula, CA (US); Gregory S. Simmons, Georgetown, TX (US)

(72) Inventors: Yunbing Wang, Sunnyvale, CA (US); Byron J. Lambert, Temecula, CA (US); Gregory S. Simmons, Georgetown, TX (US)

(73) Assignee: Abbott Cardiovascular Systems Inc., Santa Clara, CA (US)

(21) Appl. No.: 13/236,419

(22) Filed: Sep. 19, 2011

Related U.S. Application Data
(63) Continuation-in-part of application No. 12/776,329, filed on May 7, 2010, now abandoned.

(51) Int. Cl. B28B 11/12; A61L 2/20

(52) U.S. Cl. 264/145; 422/34

(57) ABSTRACT

Methods of sterilizing medical devices, particularly stents, that include a polymer with ethylene oxide. The polymer may be in the device body or a coating on the device. The method entails exposure such that the temperature of the device does not exceed the glass transition temperature of the polymer in the wet stage, that is as plasticized by the sterilant. The sterilant may include water vapor.

### CROSS-REFERENCE TO RELATED APPLICATIONS

This application is a continuation-in-part of application Ser. No. 12/776,329, filed May 7, 2010, which is incorporated by reference as if fully set forth, including any drawings, herein.

### BACKGROUND OF THE INVENTION

1. Field of the Invention

This invention relates to methods of sterilization of medical devices, and particularly stents, a type of implantable medical device. More specifically, the present invention relates to sterilization of polymeric stents with ethylene oxide at a temperature lower than the glass transition temperature of the polymeric material of the stent under the conditions of sterilization.

2. Background

The term sterilization refers to the elimination of microorganisms such as fungi, bacteria and viruses, or a reduction in the bioburden of an item where bioburden refers to the number of micro-organisms with which the item is contaminated. The degree of sterilization is typically measured by a sterility assurance level (SAL) which refers to the probability of a viable microorganism being present on a product unit after sterilization.

There are a number of sterilization procedures. The broad categories include heat, chemicals, and irradiation. An example of using heat to sterilize is autoclaving of medical instruments. Cooking or canning food is also another application of using heat for sterilization. A number of chemicals can be used for sterilization including ozone, chlorine dioxide, ethylene oxide, and hydrogen peroxide. Irradiation includes exposure to gamma rays, X rays, or an electron beam. Filtration typically involves filtering through a 0.2 micron filter.

The choice of sterilization technique will depend upon the application, and the sterility level desired. The required SAL for a product is dependent on the intended use of the product. For medical devices in particular, the level of sterility for a Class I device as per United States Food and Drug Administration (FDA) classifications, which presents a minimal risk of harm to the user and are simpler than Class II and Class III devices, will be different than the level required for a Class III device which “are usually those that support or sustain human life, are of substantial importance in preventing impairment of human health, or which present a potential, unreasonable risk of illness or injury.” (FDA)

In addition the United States FDA regulates devices with most regulations for medical devices and radiation emitting products found Title 21 of the Code of Federal Regulations (CFR) parts 800-1299. Although the FDA does provide some guidance on sterility levels, more specific information can be found in guidance documents provided by the International Organization of Standards (ISO) documents which were developed in conjunction with Association for the Advancement of Medical Instrumentation (AAMI). SALs for various medical devices can be found in materials from the AAMI in Arlington, Va.

Many medical devices undergo terminal sterilization, that is sterilization occurs in the final packaged product. Thus, the sterilization operation may have a negative impact on the material of the device, and/or any active agents. The present invention is directed to methods of sterilization that limit or eliminate some of these negative impacts on the device, and particularly methods involving ethylene oxide sterilization.

### SUMMARY OF THE INVENTION

The present invention relates to methods of sterilizing articles, more specifically medical devices including stents. The methods include, but are not limited to, exposing an implantable medical device to a fluid, and in particular a gas, comprising ethylene oxide (EtO). The device has a device body including, but not limited to, a polymer with a glass transition temperature between about 50° C. and about 60° C. During the exposure, the temperature of the device does not exceed 40° C. and does not fall below 15° C. As a result of the exposure to the fluid, such as a gas, the sterility assurance level (SAL) of the device is less than 1×10⁻⁶.

Embodiments of the present invention encompass methods which include, but are not limited to, exposing an implantable medical device, the device having a device body of biodegradable polymer, to a gas comprising EtO. During the exposure the temperature of the device is not greater than 40° C. and not less than 15° C. As a result of the exposure, the SAL of the device is less than 1×10⁻⁶ as a result of the exposure.

Encompassed in the various embodiments of the present invention are methods of fabricating a polymeric stent. These methods include, but are not limited to, the following operations: forming a tube, the tube including a polymer; cutting a stent pattern into the tube to form a polymeric stent; and exposing the polymeric stent to a gas comprising EtO such that the temperature of the stent is not greater than 40° C. and not less than 15° C. during the exposure, and such that the SAL of the device is about 1×10⁻³ or less than 1×10⁻³ as a result of the exposure to the gas. The glass transition temperature of the polymer of the device body is between about 50° C. and about 60° C.

In some embodiments, the SAL of the device is less than 1×10⁻⁶ as a result of the exposure to the gas.

In some embodiments, the polymer of the device body is poly(L-lactide) (PLLA), polymandelide (PM), poly(DL-lactide) (PDLLA), polyglycolide (PGA), poly(L-lactide-co-glycolide) (PLGA), or any block, alternating, or random copolymer thereof, or any block, alternating, or random copolymer of at least one of the foregoing group of polymers and at least one of the group of consisting of polycaprolactone (PCL), poly(trimethylene carbonate) (PTMC), polydioxanone (PDO), poly(4-hydroxy butyrate) (PHB), and poly(butylene succinate) (PBS), or any blend of the aforementioned polymers.

### BRIEF DESCRIPTION OF THE DRAWINGS

FIG. 1 depicts a stent.

FIGS. 2A-C depict a portion of a representative stent element from a typical stent.

### DETAILED DESCRIPTION OF THE INVENTION

Use of the singular herein includes the plural and vice versa unless expressly stated to be otherwise, or obvious from the context that such is not intended. That is, “a” and “the” refer to one or more of whatever the word modifies. For example, “a device” includes one device, two devices, etc. Likewise, “a polymer” may refer to one, two or more polymers, and “the polymer” may mean one polymer or a plurality of polymers. By the same token, words such as, without limitation, “devices” and “polymers” would refer to one device or polymer as well as to a plurality of devices or polymers unless, again, it is expressly stated or obvious from the context that such is not intended.

As used herein, any ranges presented are inclusive of the end-points. For example, “a temperature between 10° C. and 30° C.” or “a temperature from 10° C. to 30° C.” includes 10° C. and 30° C., as well as any temperature in between.

As used herein, words of approximation such as, without limitation, “about” “substantially,” “essentially” and “approximately” mean that the word or phrase modified by the term need not be exactly that which is written but may vary from that written description to some extent. The extent to which the description may vary will depend on how great a change can be instituted and have one of ordinary skill in the art recognize the modified version as still having the properties, characteristics and capabilities of the modified word or phrase. In general, but with the preceding discussion in mind, a numerical value herein that is modified by a word of approximation may vary from the stated value by ±15%, unless expressly stated otherwise.

As used herein, the use of “preferred,” “preferably,” or “more preferred,” and the like to modify an aspect of the invention refers to preferences as they existed at the time of filing of the patent application.

This invention relates to sterilization of medical devices, and more specifically, the present invention relates to sterilization using EtO of implantable medical devices including a polymer at a temperature lower than the glass transition temperature of polymer of the device in the “wet state,” or that is as plasticized in the sterilization procedure. The sterilization procedure is referred to as “cold” EtO sterilization as the process is performed in a manner such that the medical device temperature is about 40° C. or lower than 40° C. The novel sterilization methods of the present invention provide adequate sterilization. In some embodiments, the relative humidity may be in the range of 20% to 50%.

Implantable medical devices include appliances that are totally or partly introduced, surgically or medically, into a patient's body or by medical intervention into a natural orifice, and which are intended to remain there after the procedure. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves, closure devices for patent foramen ovale, vascular closure devices, cerebrospinal fluid shunts, and intrauterine devices.

Although the discussion that follows focuses on a stent as an example of an implantable medical device, the embodiments described herein are applicable to other implantable medical devices.

A stent is a Class III medical device per FDA medical device classifications. Stents may also be medicated, that is manufactured to also deliver an active agent (drug) to the patient. A medicated stent falls into the FDA definition (as per 21 CFR §3.2(e)) of a combination product due to the combination of an active agent (drug) or biologic with a medical device.

Stents are implantable medical devices that are generally cylindrically shaped and function to hold open, and sometimes expand, a segment of a blood vessel or other anatomical lumen such as urinary tracts and bile ducts. A “lumen” refers to a cavity of a tubular organ such as a blood vessel. Stents are often used in the treatment of atherosclerotic stenosis in blood vessels. “Stenosis” refers to a narrowing or constriction of a bodily passage or orifice. “Restenosis” refers to the reoccurrence of stenosis in a blood vessel or heart valve after it has been treated (as by balloon angioplasty, stenting, or valvuloplasty) with apparent success. In treatment of stenosis, stents reinforce body vessels and prevent restenosis following angioplasty in the vascular system. In addition to treatment for coronary artery disease such as atherosclerosis and restenosis, stents may be used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease carotid artery disease, peripheral arterial disease (PAD), and vulnerable plaque. For treatment of PAD, stents may be used in peripheral arteries such as the superficial femoral artery (SFA).

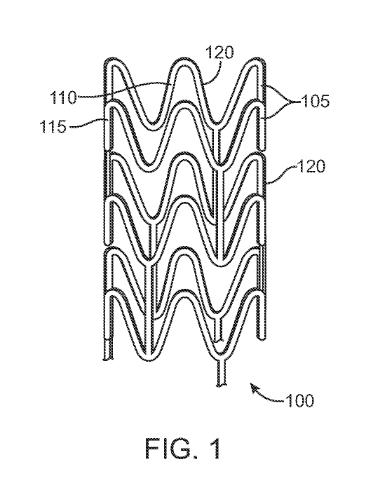

Stents are typically composed of scaffolding that physically holds open and, if desired, expands the wall of a passage way. An example of a stent 100 is depicted in FIG. 1. In some embodiments, a stent includes a scaffolding having a pattern or network of interconnecting structural elements or struts 105, which are designed to contact the lumen walls of a vessel and to maintain vascular patency, that is to support the bodily lumen. Struts 105 of stent 100 include luminal faces or surfaces 110, abluminal faces or surfaces 115, and side-wall faces or surfaces 120. The pattern of structural elements 105 can take on a variety of patterns, and the structural pattern of the device can be of virtually any design. Typical expanded dimensions of a coronary stent are 3.5-4.5 mm. In general, the body of a medical device is the main or central portion of the device. For some medical devices the device body is the device in the fully functional form before a coating or other material different from that of which the body is formed has been applied or attached. As an example, for a stent, the device body is the scaffolding. The embodiments disclosed herein are not limited to stents, or to the stent pattern, illustrated in FIG. 1.

The treatment of a diseased site or lesion with a stent involves both delivery and deployment of the stent. Typically, stents are capable of being compressed, or crimped, onto a catheter so that they can be delivered through narrow vessels to, and deployed at, a treatment site. Typical dimensions of a stent as compressed on a delivery device, such as a balloon, are about 0.04″ to 0.05″. Delivery includes inserting the stent through small lumens using a catheter and transporting it to the treatment site. Deployment includes expanding the stent to a larger diameter once it is at the desired location, that is the treatment location such as a lesion. In the case of a self-expanding stent, the stent may be secured to the catheter via a retractable sheath or a sock. When the stent is in a desired bodily location, the sheath may be withdrawn which allows the stent to self-expand. For a balloon expandable stent, the stent is expanded by inflating the balloon. The balloon may then be deflated and the catheter withdrawn.

The stent must be able to satisfy several mechanical requirements. The stent must have radial strength and sufficient strength and rigidity to support the walls of a vessel and withstand radially compressive forces. Longitudinal flexibility is required for delivery and deployment. Relatively high toughness or resistance to fracture is required for the material of the stent must be able to withstand crimping onto a delivery element as well as expansion when deployed. It must maintain its shape once deployed. For stents used in the SFA, the mechanical requirements can be higher than for stents in coronary arteries as the SFA is subjected to various forces, such as compression, torsion, flexion, extension, and contraction, which place a high demand on the mechanical performance of implants. The mechanical requirements on a stent differ from those of other implantable medical devices such as catheters, which are not crimped to a smaller size and/or expanded.

Although stents made of nonerodible metals and metal alloys have become the standard of care for treatment of artery disease, it is desirable to make stents out of biodegradable polymers. In many treatment applications, the presence of a stent in a body is necessary for a limited period of time until its intended function of, for example, maintaining luminal patency and/or active agent delivery is accomplished. Therefore, a device body, such as the scaffolding of a stent, may be fabricated from biodegradable, bioabsorbable, and/or bioerodable polymers and can be configured to partially or completely erode away after the clinical need for them has ended.

The duration during which the device maintains luminal patency depends on the bodily disorder that is being treated. For example, in treatments of coronary heart disease involving use of stents in diseased vessels, the luminal patency duration can be in a range from several months to a few years. The luminal patency duration is typically up to about 6 months, 12 months, 18 months, or two years. In some situations, the luminal patency treatment period can extend beyond 2 years. As another example, in treatments of SFA, the luminal patency duration can be in a range from 1 to 2 months to several years. For SFA, the luminal patency duration is typically up to about 6 months, 12 months, 18 months, or 2 years. Preferably luminal patency is maintained for a time period between about 6 months and about 8 months.

Although biodegradable polymers can de designed to erode away, as noted above, one drawback of polymers as compared to metals and metal alloys is that the strength to weight ratio of polymers is usually smaller than that of metals. To compensate for this, a polymeric stent can require significantly thicker struts than a metallic stent, which results in an undesirably large profile. For example, a typical thickness for a strut in a metal stent is about 0.03″.

To avoid large struts, polymers may be processed to improve strength and toughness. The embodiments of the present invention are directed to methods of sterilization in which the strength, toughness, and biodegradation rate of a polymer are not negatively impacted by the sterilization process.

An example of some of the process operations that may be involved in fabricating a polymeric stent include, but are not limited to, the following:

(1) forming a polymeric tube using extrusion or injection molding, or by rolling and welding a polymer sheet;

(2) radially and/or axially deforming (expanding and/or extending) the formed tube by application of heat and/or pressure;

(3) forming a stent from the deformed tube by cutting a stent pattern in the deformed tube such as with chemical etching or laser cutting;

(4) optionally coating the stent with a coating including an active agent;

(5) crimping the stent on a support element, such as a balloon on a delivery catheter;

(6) packaging the crimped stent/catheter assembly; and

(7) sterilizing the stent assembly.

A noted in step (2), an extruded polymer tube may also be radially expanded and/or axially extended. The tube is radially expanded to increase its radial strength, which can also increase the radial strength of the stent. The radial expansion process tends to preferentially align the polymer chains along the radial or hoop direction which is believed result in enhanced radial strength. The radial expansion step is crucial to making a stent scaffolding with thin struts that is sufficiently strong to support a lumen upon implantation. The tube at both the initial and expanded diameter have wall thicknesses that are large enough that they can support an outward radial force or load. In contrast a tubular membrane structure, such as a balloon, has a wall thickness that is so thin that the tubular membrane cannot support a load at a given diameter unless it is preloaded, i.e., inflated with a fluid, such as a gas.

During the expansion step, the tube is heated to a temperature between glass transition temperature (T_{g}), and the melting point of the polymer and the tube is expanded to an expanded diameter. Upon expansion the tube is cooled to below the Tg of the polymer, typically to ambient temperature, to maintain the tube at an expanded diameter. The percent radial expansion may be between about 200% and 500%, preferably 400% to 500%, or any specific value within either of these ranges. The percent radial expansion is defined as RE %=(RE ratio−1)×100%, where the RE Ratio=(Inside Diameter of Expanded Tube)/(Original Inside Diameter of the tube). The percent of axial extension that the polymer tube undergoes is defined as AE %=(AE ratio−1)×100%, where the AE Ratio=(Length of Extended Tube)/(Original Length of the Tube). The percent axial extension expansion may be between about 20% and about 200%, preferably about 20% and about 120%, or any specific value within either of these ranges. The width and thickness of the struts of the stent can be, for example, between 100-160 microns.

After cutting a stent pattern into the expanded tube, as noted in step (4) the stent scaffolding may then be optionally coated with a drug delivery coating which can include a polymer and an active agent. The active agents may be distributed uniformly or non-uniformly in a coating that is disposed over all of, substantially all of, or at least a portion of, the outer surface of the device. The “outer surface” of a device is meant any surface however spatially oriented that is in contact with bodily tissue or fluids. For a stent, the outer surface includes the abluminal surface, the luminal surface, and the sidewall surfaces. Alternatively and/or additionally, if the structural elements, such as a strut or a portion of a strut on a stent, are manufactured from a polymer, or include a continuous phase of a polymer (about 20% or about 25% by volume polymer or more), active agents may be distributed uniformly, or non-uniformly, throughout the polymer of the structural elements.

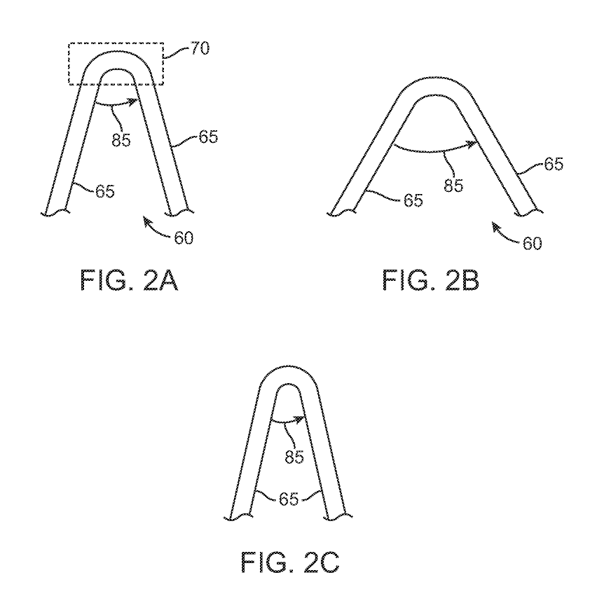

In order to make the stent ready for delivery, the stent is secured to a delivery element such as a delivery balloon. In this process, the stent is compressed to a reduced diameter or crimped over the balloon. During crimping and in the crimped state, the crowns of the stent are subjected to high, localized stress and strain. FIG. 2A depicts a partial planar side view of a luminal or abluminal surface of a portion 60 from a stent in an unexpanded state that includes straight sections 65 and a curved section 70, also called a crown, with an angle 85 between straight sections 65. When a stent undergoes radial expansion, portions of struts bend resulting in an increase of an angle 85, as shown in FIG. 2B. As shown in FIG. 2C, when a stent is crimped, angle 85 decreases. Typically, from crimping to expansion the strut may be bent from an angle of about 30° to an angle of about 120°, and/or through a range of 100° to 150°. Due to the fact that the inside or concave region of the crowns is subjected to high compressive stress and strain, as shown in FIG. 2C, the stent during crimping and in the crimped state is susceptible to cracking.

The stent is deployed by expanding it to an increased diameter at an implant site in a vessel which can be greater than the as-cut diameter of the stent. The deployed stent must have sufficient radial strength to apply an outward radial force to support the vessel at an increased diameter for a period of time. The crown regions of the deployed stent are under high stress and strain during expansion and after deployment.

Due to the high stress and strain the stent is subjected to during use, it is important for the stent body to have high fracture toughness to inhibit cracking. Fracture toughness is enhanced for a semi-crystalline polymer by minimizing the size of crystalline domains and achieving an optimal amorphous/crystalline ratio. The crystallinity provides strength and stiffness (high modulus) to the polymer which is needed for supporting a vessel. However, if the degree of crystallinity is too high, the polymer may be too brittle and is more susceptible to fracture. The degree of crystallinity for a PLLA scaffolding may be about 20% to about 50%.

Since crystals nucleate and grow between T_{g} and the melting temperature of a polymer, the size of crystalline domains and degree of crystallinity depend on process parameters of the radial expansion process, such as the expansion temperature, heating rate, and time spent above T_{g}. Generally, smaller crystals are favored or generated at lower temperatures closer to T_{g} than the melting temperature. For example, for a polymer tube of PLLA with a T_{g} of about 58° C., an expansion temperature of 65-120° C. is preferred. In some embodiments the expansion temperature may be 65° C.-90° C., or 65° C.-85° C.

In general, it is crucial to inhibit loss of properties generated by the radial expansion in later processing steps and after manufacture during a storage period all the way to the deployment of the stent in a patient. These properties include alignment of polymer chains, the small crystalline domains, and the degree of crystallinity. Exposure of the stent to temperatures above T_{g} may modify these properties and could negatively impact the performance of the stent when implanted. It is believed that exposure to the stent to temperatures above T_{g} for even short durations, such as 3 to 5 minutes, may negatively impact the mechanical properties.

As noted in step (7) the stent is typically terminally sterilized. In other words, the stent is sterilized after it has been manufactured, optionally coated, mounted on a delivery device, covered with a sheath if required, attached to a delivery system, and packaged. However, in some embodiments, the stent may be sterilized at an earlier stage of manufacture. Many of the currently used sterilization techniques may potentially impact the polymer properties and/or active agent properties. Autoclaving, which is often used for surgical instruments, is not used for stents due to the high temperature, and exposure to humidity. Similarly, the high temperatures and humidity in standard EtO sterilization may present problems. It is known that radiation may impact polymers and/or active agents. For a polymer stent, changes to polymer molecular weight, resulting in changes to mechanical properties and polymer degradation rate, may potentially occur even though the product is sterilized using low doses of radiation (25 kGy). Because the safety and efficacy of bioabsorbable stents are strongly dependent on mechanical properties such as radial strength, recoil, and crack resistance, clearly any change in these properties during sterilization is undesirable.

The present invention is directed to methods of sterilization of medical devices including polymers, and in particular, stents formed of or including polymers, that limit, or eliminate the negative impact of sterilization on the properties of the device. Specifically, the mechanical properties of the device, in particular the fracture toughness and radial strength, as well as and the purity and content of any active agents, are preserved, or substantially preserved. Additionally, the methods limit or eliminate any negative impact of sterilization on the degradation rate of a bioabsorbable polymer.

It has surprisingly been found that in order to obtain a polymeric stent which does not suffer from fracture when deployed after EtO sterilization, that the EtO sterilization process has to be performed at a temperature lower than conventional EtO sterilization and with tighter control of temperature excursions. For PLLA stents, the sterilization occurs at about 40° C. or lower than 40° C., preferably about 35° C. or lower than 35° C., and more preferably about 32° C. or lower than 32° C., and in some embodiments, about 30° C. or lower than 30° C., or about 28° C. or lower than 28° C.

For stents manufactured from one or more polymers, conventional wisdom is that as long as the sterilization temperature is lower than its T_{g}, the mechanical properties of the stent would not be impacted by sterilization. However, it was surprisingly found that the mechanical properties remained unchanged or substantially unchanged only when the device was sterilized at a temperature much lower than its T_{g}. It is also believed that a low humidity during the sterilization procedure may help maintain the mechanical properties.

In this application, embodiments include methods of sterilizing polymeric devices, and in particular those made from biodegradable polymers, such as biodegradable polyesters including, without limitation, PLLA, PGA, and PLGA, with EtO such that the temperature of the device does not exceed 40° C., preferably 35° C., more preferably 32° C., and even more preferably 30° C., and in some embodiments, the temperature of the device does not exceed 28° C.

Conventionally, EtO sterilization processes are run at a temperature of about 50° C.-60° C. or higher. A trial of with EtO sterilization of PLLA stents was carried out at 60° C., which is slightly above the Tg of PLLA of 58° C. The results from the EtO sterilization at 60° C. did not appear to be promising.

This may be explained since above the T_{g} the polymer chains have more freedom of movement and may relax. As noted above, even limited duration of exposure to temperatures above the T_{g} would change the micro structural properties. Therefore, a sterilization process that exposes the stent to a temperature above the T_{g} is unfavorable. The micro structural properties developed in the previous manufacturing processes, and particularly as a result of radial expansion to provide radial strength and fracture toughness, can be modified. The micro structural properties include the chain alignment/orientation, degree of crystallinity, the small crystal size. Above T_{g}, the chain alignment/orientation would be partially or totally lost, and therefore fracture toughness would be lost. It also potentially possible that the percent crystallinity and crystal size change as a result of solvent-induced crystallization that may potentially occur during the sterilization process at these higher temperatures and humidity values.

In a subsequent trial, PLLA stents were sterilized with EtO using an 8 ft³ Research and Development (R & D) chamber at a chamber setting of 40° C. Temperature measurements indicated that the product in the chamber reached a temperature of about 50° C. even though the chamber was set to 40° C. Exposure to a temperature of 50° C., for the duration of sterilization, was known to be acceptable for the active agent. Although the sterility resulting from sterilization with EtO under these circumstances was acceptable, it was very surprisingly found that many of the stents exhibited broken struts upon deployment. The high number of broken stents was surprising as the temperature utilized was below the T_{g} of PLLA, that is below 58° C.

The next trial used also used the 8 ft³ Research and Development (R & D) chamber In this trial, the product temperature more closely matched the chamber set-point temperature. PLLA stents were sterilized with EtO in the R & D chamber set to 30° C. and the devices were close to a temperature of 30° C. (about 32° C.) during sterilization. None of the stents that were subjected to EtO sterilization at 30° C. exhibited fractured struts on deployment.

Without being bound by theory, it is believed that the mechanical properties of the stent are impacted by plasticization by both water (in the form of humidity) and EtO. Plasticization of a polymer refers to the addition of a second, lower T_{g} and generally lower molecular weight, material such as an organic liquid or solvent to a polymer. The effect is to lower the T_{g} of the polymer (actually the T_{g} measured is that of the polymer and the plasticizer but conventionally is referred to as the T_{g} of the polymer), and generally, also to transform a hard, brittle material to a soft, rubber-like material. Not all lower T_{g} materials will act as a plasticizer for a particular polymer because the two materials must be compatible and have some mutual solubility. It is believed that because of the plasticization due to the humidity and the EtO in the EtO sterilization, the T_{g} of the polymer is lowered such that the product temperature is at or above T_{g}. When the polymer is at or above T_{g} the polymer chains move more freely, and thus the polymer chain orientation may be partially or substantially lost due to stress relaxation. As noted above, it is believed that even a short duration of time above the T_{g} of the polymer may have a negative impact on the polymer micro structural properties. These micro structural properties include alignment of polymer chains and morphology. It is potentially possible that the crystallinity changes as well.

To confirm this hypothesis, PLLA stents were heated at 50° C. in an oven for an hour before sterilization was performed in the R & D chamber set to 30° C. The polymeric stents from this trial did not exhibit any broken struts. This result supports the theory that the plasticization by the humidity and the EtO in the chamber results in a lower glass transition temperature of the polymer during the sterilization process since the mechanical properties remained unchanged when exposed to 50° C. temperature in the absence of humidity and EtO. The T_{g} of PLLA in a humid environment is about 42° C., and the T_{g} of the PLLA could potentially be lower than this in the sterilization chamber due to potential plasticization by the EtO.

Thus, the embodiments of the present invention are directed to methods of sterilization with EtO at lower than conventional temperatures. Specifically, the methods control the temperature of the device such that it is at, and preferably, below the glass transition temperature of the polymer as plasticized by the EtO and humidity.

A non-limiting example of a standard, conventional, or typical EtO sterilization procedure includes the following operations:

1) a vacuum is pulled to remove air, often to about 80 mm HgA (mmHg Absolute) or less;

2) the chamber is humidified to an initial level, and the chamber is pulsed with steam which in some cases brings the chamber to about 30%-70% relative humidity;

3) the chamber is allowed a “humidity dwell” time of about 1-5 hours to allow the water vapor to at least partially penetrate the product;

4) EtO is injected into the chamber, often followed by a nitrogen blanket to remove or limit air (for safety reasons);

5) The chamber is held at a specified temperature and pressure with the EtO present;

6) An aeration operation in which the chamber is repeatedly pulsed with steam, nitrogen, and air to remove the EtO;

7) A final “wash” of the chamber is performed with air at atmospheric pressure.

The above description is typical for a dynamic pulsed steam EtO procedure that is entirely run within the EtO chamber. Alternatively, the humidification can occur in a static relative humidity (RH) preconditioning room and aeration can occur post sterilization in an forced air aeration room.

For cycles that include steam pulsing, the steam that is introduced may be at about 75 mmHg. A typical steam pulse is about 15 mm HgA and takes about 10 minutes per pulse cycle. In a typical procedure, there are approximately 6 steam pulses. EtO injection may be accomplished by heating liquid EtO, and then lowering the pressure such that the EtO vaporizes. In practice, high pressure and slightly heated EtO is injected into the chamber which is at a lower pressure, with subsequent vaporization of the EtO. The level of EtO is about 500 mg/L. The hold time once the EtO is injected may be from 1-10 hours. For aeration of the EtO, in cycles with dynamic aeration, the steam/nitrogen/and air pulse takes the chamber from vacuum to a higher pressure, with the initial pulses being primarily nitrogen and more air being introduced as the number of pulses progresses. There may be approximately 15 cycles for aeration. Other methods of aeration may be used.

The specific pressure set points for the chamber during the steam and EtO injections are specific for each chamber. Thus, the parameters above may be adjusted depending upon the chamber used to obtain a desired level of humidity and a desired concentration of EtO. These parameters are examples of typical values, and values other than the above specified values are possible.

It has been found that the key to maintaining the tight control of the temperature in the EtO sterilization process is the careful choice of the steam pulse cycling during the humidification step. The length of the humidity injection, the amount of vapor added during the injection (typically measured as a pressure differential in the chamber), the time between pulses, and the total number of pulses all interact. A design of experiments was used to determine the processing parameters that would allow the device temperature to remain within a desired temperature range, that is about 40° C. or lower than 40° C., preferably about 35° C. or lower than 35° C., more preferably about 35° C. or lower than 35° C., about 32° C. or lower than 32° C., or still even more preferably about 30° C. or lower than 30° C.

To determine the actual cause and solve the problem of strut breakage, it was necessary to measure the actual product temperature. As noted above, for the 40° C. EtO sterilization, the temperature of the product differed from the temperature “set-point” of the chamber. The “set-point” is the temperature dialed in the chamber temperature controller. It is believed that the deviation of the product temperature from the chamber set-point temperature is due to condensation of steam on the product. If one had relied upon the “set-point” of the chamber one would not have discovered the problem. The successful trial at 30° C. was performed in a commercially available R & D chamber. However, the chamber had been retrofitted with an improved controller and additional temperature and humidity probes. The custom modified R & D chamber provided better data reporting capabilities than the “as received” commercially available chamber. It was the careful monitoring of the actual process that provided the data to identify the issue, and allowed for better, and tighter control of the temperature. Thus, use of the off-the-shelf EtO sterilization chamber according to the manufacturer's instructions, or in the conventional manner, would not have allowed one to have identified the problem and to have developed a solution. Although commercially available and/or contract facilities monitor and validate the product temperature, these facilities may potentially be unable to obtain tight temperature control, and/or tight humidity control.

Various embodiments of the present invention include methods of EtO sterilization of stents comprising a polymer in which the sterilization does not negatively impact the fracture toughness, the radial strength, and the biodegradation rate of the polymer. Included are methods of sterilizing an implantable medical device, that is achieving a specified SAL, by exposing the device, the device comprising a device body comprising a polymer with a glass transition temperature of between about 50° C. to about 60° C., to a gas comprising EtO such that during the EtO gas exposure phase of the cycle the device is at a temperature not greater than 40° C. and not less than 15° C. In some embodiments, the device temperature is not greater than 40° C. and not less than 15° C. during the entire EtO sterilization cycle. The unplasticized glass transition temperature of the polymer is the glass transition temperature prior to sterilization, or in other words, when not plasticized by EtO and humidity.

The embodiments of the present invention encompass exposure to a fluid comprising EtO, where a fluid may be a liquid, gas or vapor, or supercritical fluid. However, in preferred embodiments, the exposure is to a gas including EtO. Thus, although embodiments of this invention may refer to exposure to a gas including EtO, in any of these embodiments, the exposure may be to a fluid comprising EtO. The exposure to the gas may be performed by utilizing substantially pure liquid EtO (100% EtO) which is vaporized. Exposure may also be performed by placing the device in a chamber or container and filling the container with a gas comprising 100% EtO, or EtO mixed with diluents, e.g., hydrochlorofluorocarbons (HCFCs). The gas in the container or chamber may be stagnant, or more commonly may be circulating. The chamber or container may have a flow of gas through the chamber. The exposure may be any combination of the above.

The lower limit for the EtO concentration may be not less than 300 mg/liter, not less than 350 mg/liter, or not less than 400 mg/liter. The upper limit of the EtO concentration in the gas may be not more than 1,000 mg/liter, not more than 900 mg/liter, not more than 800 mg/liter, or not more than 600 mg/liter. Embodiments of the invention encompass the ranges of EtO concentration of any combination of the above recited upper and lower limits. As an example, in a preferred embodiment, the EtO concentration range may be not less than 400 mg/liter and not more than 600 mg/liter. The gas may also include other substances, such as nitrogen, other inert gases, and/or HCFCs. In some embodiments the gas also includes water vapor, or humidity. The lower limit of the relative humidity may be 15%, 25%, or 30%. The upper limit of the relative humidity may be 50%, 60%, or 80%. Thus, embodiments of the present invention encompass a relative humidity range that is any combination of the upper and lower limits recited above. In some embodiments, a relatively low humidity between about 20% and about 50%, between about 20% and about 30%, above 15% but lower than 30%, between about 15% and about 30%, between about 15% and about 25%, above 15% but lower than 25%, between about 10% and about 25%, between about 10% and about 22%, or above 8% but lower than 22% may be used to minimize any negative impact on the mechanical properties and the degradation profile of the stent. In still other embodiments, the humidity may be maintained within about 16% and about 29%, about 17% and about 28%, about 18% and about 27%, about 16% and about 26%, about 20% and about 25%, and about 12% and about 25%. In some embodiments, the humidity may be maintained within any of the above ranges for substantially all of the duration of the EtO exposure , for at least 97% of the duration of the EtO exposure with any excursions not to exceed 45%, for at least 95% of the duration of the EtO exposure and with any excursions not to exceed 45%, for at least 90% of the duration of the EtO exposure with any excursions not to exceed 45%, or for any of the previous time frames but the duration including not only the duration of EtO exposure but also including all additional time up to the time that aeration operation is initiated. Conventional wisdom holds that the humidity should be 30% or higher to obtain sufficient microbial kill. The gas composition may change with time. Oxygen is often eliminated to avoid explosive scenarios, and in particular in those embodiments in which HCFC diluents are not utilized. In some embodiments, the EtO chamber includes essentially no oxygen (less than 100 mg/liter or less than 25 mg/liter), while in other embodiments the elimination of oxygen is not complete.

The duration of the exposure is a function of the concentration of the EtO, the desired SAL, the temperature of exposure, the temperature of the device, and other factors. In some embodiments the duration is from 1 hour to 20 hours, preferably from 1 hour to 10 hours, and even more preferably from 1 hour to 6 hours. The duration of exposure refers to the time frame during which the device is exposed to a gas including EtO of not less than a minimum level, such as 300 mg/liter, and does not mean the duration of the entire sterilization cycle. The EtO concentration may fluctuate during the exposure. The duration may include two or more time periods of exposure to a gas including EtO of not less than a minimum level separated by time during which the gas includes less than the minimum level. In some embodiments, the duration is from the time of EtO injection to the start of the aeration operation, that is the operation to remove the EtO. The minimum level may be 300 mg/liter, preferably 350 mg/liter, or more preferably 400 mg/liter.

Various embodiments of the present invention encompass exposure that results in a SAL of about 1×10⁻³ or lower than 1×10⁻³, preferably about 1×10⁻⁴ or lower than 1×10⁻⁴, and more preferably about 1×10⁻⁶ or lower than 1×10⁻⁶. Exposure to a gas including EtO is an exposure sufficient to sterilize a device, that is sufficient to obtain a SAL of at least 1×10⁻³ or lower, or within a specified limit. The SAL level obtained is a function of the duration of exposure, concentration of EtO, size and shape of the devices to be sterilized, as well as the temperature, pressure, and humidity under which the exposure occurs. Thus, incidental exposure to an environment in which EtO is present is not likely to result in sterilization.

Various embodiments of the invention encompass exposure wherein the temperature of the device is not greater than 40° C. and not less than 10° C., not greater than 40° C. and not less than 15° C., not greater than 40° C. and not less than 20° C., not greater than 40° C. and not less than 25° C., not greater than 40° C. and not less than 25° C., not greater than 40° C. and not less than 28° C., not greater than 40° C. and not less than 30° C., not greater than 38° C. and not less than 15° C., not greater than 38° C. and not less than 10° C., not greater than 38° C. and not less than 20° C., not greater than 38° C. and not less than 25° C., not greater than 38° C. and not less than 30° C., not greater than 38° C. and not less than 32° C., not greater than 35° C. and not less than 10° C., not greater than 35° C. and not less than 15° C., not greater than 35° C. and not less than 20° C., not greater than 35° C. and not less than 25° C., not greater than 35° C. and not less than 30° C., not greater than 32° C. and not less than 10° C., not greater than 32° C. and not less than 15° C., not greater than 32° C. and not less than 20° C., not greater than 32° C. and not less than 25° C., not greater than 32° C. and not less than 28° C., not greater than 30° C. and not less than 10° C., not greater than 30° C. and not less than 15° C., not greater than 30° C. and not less than 20° C., not greater than 30° C. and not less than 25° C., not greater than 28° C. and not less than 25° C., and not greater than 28° C. and not less than 22° C., not greater than 28° C. and not less than 20° C., not greater than 28° C. and not less than 15° C., not greater than 28° C. and not less than 10° C., not greater than 25° C. and not less than 20° C., not greater than 25° C. and not less than 18° C., not greater than 25° C. and not less than 15° C., not greater than 25° C. and not less than 10° C., not greater than 26° C. and not less than 18° C., not greater than 26° C. and not less than 15° C., not greater than 26° C. and not less than 20° C., not greater than 24° C. and not less than 16° C. not greater than 24° C. and not less than 12° C., and not greater than 24° C. and not less than 10° C. during the time of EtO injection and EtO exposure. In addition to embodiments in which the temperature of the device is maintained within one of the above ranges during the exposure to the EtO, but other embodiments are included in which the temperature of the device is maintained within any of the ranges recited above for the duration of the entire sterilization cycle, and still other embodiments where the duration is up to the time that an aeration operation is initiated.

Various embodiments of the invention encompass exposure wherein the temperature of the device does not exceed the glass transition temperature, does not exceed 3° C. less than the glass transition temperature, does not exceed 5° C. less than the glass transition temperature, and does not exceed 10° C. less than the wet glass transition temperature of the polymer of the device, that is the glass transition temperature as plasticized by the fluid. The term “wet” glass transition temperature or “as plasticized glass transition temperature” refers to the T_{g} of the polymer plasticized by the fluid, or gas, in the sterilization chamber.

This means the level of plasticization achieved under the conditions of the exposure. Thus, the “as plasticized” T_{g} or “wet” T_{g} does not necessarily require the maximum plasticization possible, or in other words, it does not require that the polymer contains the equilibrium concentration of gas, fluid, and/or humidity, although this may be the situation. Also, the level of plasticization, and thus the glass transition temperature, may change over time during the exposure. Conversely, when the term T_{g} is used without descriptors “wet” or “as plasticized,” then the T_{g} is that of the polymer as measured before placement in the EtO sterilization chamber. As non-limiting examples, the T_{g} in a humid environment, which is close to the wet T_{g}, for a stent of PLLA is about 40° C., and for a stent of PLGA with 15% GA content, the T_{g} in a humid environment is about 35° C.

Various embodiments of the invention encompass methods of sterilization in which the steam pulsing and other process parameters are controlled such that the temperature of the device does not deviate from the “set-point” temperature for the chamber or by more than 5° C., by more than 3° C., by more than 2° C., or by more than 1.5° C. for substantially all of the time, about 95% or more of the time, or about 90% or more of the time that the device is exposed to EtO (EtO dwell time), of the time up to the sterilization cycle, or of the time of the entire sterilization cycle.

Embodiments of the invention encompass methods of sterilization of a medical device including a polymer with a gas comprising EtO such that after sterilization, the fracture toughness of the device, and/or the fracture toughness of the polymer of the device, is about 70% of the initial fracture toughness or greater than 70% of the initial fracture toughness, about 80% of the initial fracture toughness or greater than 80% of the initial fracture toughness, and about 90% of the initial fracture toughness or greater than 90% of the initial fracture toughness.

Embodiments of the invention encompass sterilization of a medical device including a polymer such that after sterilization, the modulus of the device and/or the modulus of the polymer of the device, is about 70% of the initial modulus or greater than 70% of the initial modulus, about 80% of the initial modulus or greater than 80% of the initial modulus, and about 90% of the initial modulus or greater than 90% of the initial modulus. The above embodiments encompass a Young's modulus, a compressive modulus, a bulk modulus, and a shear modulus.

Embodiments of the invention encompass sterilization of a stent comprising a polymer, such that after sterilization, the hoop strength of the stent is about 70% of the initial hoop strength or greater than 70% of the initial hoop strength, about 80% of the initial hoop strength or greater than 80% of the initial hoop strength, and about 90% of the initial hoop strength or greater than 90% of the initial hoop strength.

Embodiments of the invention encompass sterilization of a stent comprising a polymer, such that after sterilization, the percentage of stents exhibiting broken struts upon deployment to the nominal diameter is about 20% or lower than 20%, about 15% or lower than 15%, about 10% or lower than 10%, about 5% or lower than 5%, and about 2% or lower than 2%.

The present invention relates to methods of sterilization of medical devices including polymers. In some embodiments, the device body, or scaffolding, comprises a polymer. In some embodiments, the device body is at least 20%, or at least 50% by volume polymer. In other embodiments, the device body is at least 20% or at least 50% by weight polymer. In still other embodiments, a polymer forms a continuous phase of the device body. In some embodiments, the device body essentially consists of, or substantially consists of (about 90% or greater than 90% by volume) polymer. In some embodiments, the polymer is in a coating on the outer surface of the body of the device where the body of the device may be substantially free of polymer, or the body may include a polymer. In the embodiments including a coating, the coating may cover all, substantially all, or at least a portion of the outer surface of the device. The coating may consist essentially of a polymer and an active agent at an active agent: polymer weight ratio from about 3:1 to about 1:5, preferably from about 2:1 to about 1:3. The device body and/or a coating may optionally include, in addition to a polymer, other materials such as fillers, plasticizers, mold release agents, lubricants, stabilizers, anti-oxidants, active agents, additives to enhance radiopacity, or other additives.

The embodiments of the invention are methods of sterilizing a medical device including a polymer. The upper end of the range of the glass transition temperature of the polymer may be not more than 80° C., not more than 75° C., not more than 70° C., not more than 65° C., not more than 60° C., not more than 55° C., or not more than 50° C. The lower end of the range of the glass transition temperature of the polymer may be not less than 35° C., not less than 37° C., not less than 38° C., not less than 40° C., not less than 42° C., not less than 45° C., and not less than 50° C. Embodiments of the present invention encompass methods of sterilization in which the glass transition temperature of the polymer of the device may be within any range resulting from the combination of any lower limit and any upper limit of the glass transition temperature recited above.

For a polymeric stent, or other medical device, the glass transition temperature refers to that of the relevant polymer. For a polymeric stent the relevant polymer is the polymer, or the block of a block copolymer, that is glassy, and also makes up most of the device body, or at least a continuous phase of the device body, and which also provides the support to the body lumen. In other words, as used herein, a glassy polymer refers to a polymer having a glass transition temperature of at least 35° C. or greater than 35° C., preferably at least 40° C. or greater than 40° C., more preferably at least 45° C. or greater than 45° C., and even more preferably at least 50° C. or greater than 50° C. The device body may comprise a polymer blend. One of skill in the art is able to determine the relevant glass transition temperature(s) of the polymer of the device based upon the disclosure herein.

In some embodiments, the polymer chains of the medical device body are oriented. In preferred embodiments, the polymer is not cross-linked (or un-cross-linked), but in other embodiments, the polymers is cross-linked. If cross-linked, the polymer is preferably lightly (about 5% or less) cross-linked.

In some embodiments, the device body includes a biodegradable polymer, preferably, a biodegradable polyester, and more preferably a lactic acid based polymer, a glycolic acid based polymer, or a polymer based on both lactic and glycolic acid, such as, without limitation, PLLA, PGA, and/or PLGA, and either all or substantially all of the device body is polymer, or at least 90% by volume is polymer. In still other embodiments, the device body of any embodiment of the previous sentence or any other embodiment described herein is additionally coated with a coating of poly(D,L-lactide) and an active agent at an active: polymer ratio from about 2:1 to about 1:3. In a preferred embodiment, the device body is at least 90% by volume PLLA, PGA, and/or PLGA, and the device is coated with a coating of PDLLA and everolimus at a 1:1 weight/weight ratio. In another preferred embodiment, the device body is at least 90% by volume PLLA, and the device is coated with a coating of PDLLA and everolimus at a 1:1 weight/weight ratio.

It is believed that the methods of sterilization encompassed by the embodiments of the present invention are applicable to any polymer capable of exhibiting increased strength and mechanical properties when subjected to deformation. Thus, although the title refers to biodegradable polymers, the embodiments of the invention are not so limited, and biostable polymers are also encompassed in the embodiments of the invention. Also included are any semicrystalline polymers that are susceptible to strain induced crystallization. Biodegradable polymers are preferred, and more preferred polymers are semi-crystalline biodegradable polyesters. In some embodiments, the polymers may have a weight-average molecular weight, determined post-sterilization or after the last stage of manufacture, of about 100 Kg/mol to about 1,000 Kg/mol, preferably about 125 Kg/mol to about 600 Kg/mol, or more preferably about 150 Kg/mol to about 400 Kg/mol.

Examples of polymers that may be used in the various embodiments of the present invention include, but are not limited to: poly(lactide) in which the lactide is any one or any combination of L-lactide, D-lactide and meso-lactide, poly(L-lactide) (PLLA), polymandelide (PM), poly(DL-lactide) (PDLLA), polyglycolide (PGA), poly(lactide-co-glycolide) in which the lactide is any one or any combination of L-lactide, D-lactide and meso-lactide, poly(L-lactide-co-glycolide) (PLGA), and random, alternating, or block copolymers thereof. In some embodiments, the medical device such as a stent body, or a tube preform that is formed into a stent can be made of a random, alternating, or block copolymer of any one or more of the above polymers, and one or more of the following: polycaprolactone (PCL), poly(trimethylene carbonate) (PTMC), polydioxanone (PDO), poly(4-hydroxy butyrate) (PHB), and poly(butylene succinate) (PBS). Blends of any of the above recited polymers may be used.

The PLGA used can include any molar ratio of L-lactide (LLA) to glycolide (GA). In particular, the stent or tube may be made from PLGA with a molar ratio of (LLA:GA) including 85:15 (or a range of 82:18 to 88:12), 95:5 (or a range of 93:7 to 97:3), or commercially available PLGA products identified as having these molar ratios.

TABLE 1
Glass transition temperatures of polymers.

| | Glass-Transition |
|---|---|
| Polymer | Temp (° C.)¹ |
| PGA | 35-40 |
| PLLA | 55-60 |
| PDLLA | 55-60 |
| 85/15 PLGA | 50-55 |
| 75/25 PLGA | 50-55 |
| 65/35 PLGA | 45-50 |
| 50/50 PLGA | 45-50 |

¹Medical Plastics and Biomaterials Magazine, March 1998.

As used herein, the terms poly(D,L-lactide), poly(L-lactide), poly(D,L-lactide-co-glycolide), and poly(L-lactide-co-glycolide) are used interchangeably with the terms poly(D,L-lactic acid), poly(L-lactic acid), poly(D,L-lactic acid-co-glycolic acid), and poly(L-lactic acid-co-glycolic acid), respectively.

Active agents (drugs), may optionally be included in the device. The active agent may be in the body of the implantable medical device, and/or may be in a coating on the device. These active agents can be any agent which is a therapeutic, prophylactic, or a diagnostic agent, or any agent that is used to treat a disease or condition. Examples include, without limitation anti-restenosis, pro- or anti-proliferative, anti-inflammatory, anti-neoplastic, antimitotic, anti-platelet, anticoagulant, antifibrin, antithrombin, cytostatic, antibiotic, anti-enzymatic, anti-metabolic, angiogenic, cytoprotective, angiotensin converting enzyme (ACE) inhibiting, angiotensin II receptor antagonizing and/or cardioprotective active agents.

Some specific, but non-limiting, examples of active agents include dexamethasone, rapamycin (sirolimus), Biolimus A9 (Biosensors International, Singapore), deforolimus, AP23572 (Ariad Pharmaceuticals), tacrolimus, temsirolimus, pimecrolimus, novolimus, zotarolimus (ABT-578), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazolylrapamycin, and 40-epi-(N1-tetrazolyl)-rapamycin.

As used herein, the following definition apply:

“Sterility Assurance Level” (SAL)—refers to the probability of a viable microorganism being present on a product unit (that is the product is not sterile) after the product has undergone a sterilization procedure, or in other words, the probability that a particular unit is non-sterile after the product has undergone a sterilization procedure. Thus, a product is more sterile or has a higher sterility as the SAL numerically decreases. In other words a product with an SAL of 10⁻⁶ is more sterile than a product with an SAL of 10⁻⁴.

“Sterilize” or “sterilization”—the process by which the bioburden of an item is reduced to a particular sterility assurance level where the sterility assurance level required will depend upon the use of the article.

As used herein, a “polymer” refers to a molecule comprised of repeating “constitutional units.” The constitutional units derive from the reaction of monomers. As a non-limiting example, ethylene (CH₂═CH₂) is a monomer that can be polymerized to form polyethylene, CH₃CH₂(CH₂CH₂)ₙCH₂CH₃ (where n is an integer), wherein the constitutional unit is —CH₂CH₂—, ethylene having lost the double bond as the result of the polymerization reaction. A polymer may be derived from the polymerization of several different monomers and therefore may comprise several different constitutional units. Such polymers are referred to as “copolymers.” The constitutional units themselves can be the product of the reactions of other compounds. Those skilled in the art, given a particular polymer, will readily recognize the constitutional units of that polymer and will equally readily recognize the structure of the monomer from which the constitutional units derive. Polymers may be straight or branched chain, star-like or dendritic, or one polymer may be attached (grafted) onto another. Polymers may have a random disposition of constitutional units along the chain, the constitutional units may be present as discrete blocks, or constitutional units may be so disposed as to form gradients of concentration along the polymer chain. Polymers may be cross-linked to form a network.

As used herein, a polymer has a chain length of 50 constitutional units or more, and those compounds with a chain length of fewer than 50 constitutional units are referred to as “oligomers.” Preferably polymers used in the methods of the present invention have a weight average molecular weight of above 40,000 Daltons, preferably above 50,000, and even more preferably above 60,000 Daltons.

“Strength” refers to the maximum stress along an axis which a material will withstand prior to fracture. The ultimate strength is calculated from the maximum load applied during the test divided by the original cross-sectional area.

“Modulus” may be defined as the ratio of a component of stress or force per unit area applied to a material divided by the strain along an axis of applied force that results from the applied force. The modulus typically is the initial slope of a stress—strain curve at low strain in the linear region. For example, a material has a tensile, a compressive modulus, and a storage or shear modulus.

“Toughness” is the amount of energy absorbed prior to fracture, or equivalently, the amount of work required to fracture a material. One measure of toughness is the area under a stress-strain curve from zero strain to the strain at fracture. The units of toughness in this case are in energy per unit volume of material.

As used herein, the terms “biodegradable”, “bioerodable”, and “bioabsorbable” as well as degraded, eroded, and absorbed, when used in reference to polymers, coatings, devices, or other materials referenced herein, are used interchangeably, and refer to polymers, coatings, devices, and materials that are capable of being completely or substantially completely, degraded, dissolved, and/or eroded over time when exposed to physiological conditions (pH, temperature, and fluid or other environment), and can be gradually resorbed, absorbed and/or eliminated by the body, or that can be degraded into fragments that can pass through the kidney membrane of an animal (e.g., a human). Conversely, a “biostable” polymer, coating, or material, refers to a polymer, coating or material that is not biodegradable.

The glass transition temperatures, T_{g}, is the temperature at which a substance, which is in an amorphous phase changes mechanical properties from those of a rubber (i.e., elastic) to those of a glass (brittle), when observations of material properties are made on the same time scale. Although lower molecular weight materials, that is non-polymeric materials, may have a “glass transition temperature” which is observable if super cooled below the freezing point without crystallizing, the term is most often used with polymers, or polymer segments such as a block of a block copolymer. Below the T_{g} the molecules have very little rotational or translational freedom, i.e., they are unable to rotate or move easily or very far in relation to one another. Rather than moving around to adapt to an applied stress, they tend to separate violently so that the polymer breaks or shatters similarly to a pane of glass that is stressed. Above T_{g}, relatively facile segmental motion becomes possible and the polymer chains are able to move around and slip by one another such that when a stress is applied to the polymer it bends and flexes rather than breaks. The measured T_{g} is dependent on the heating rate, if Differential Scanning calorimetry is used for measurement, and is influenced by the thermal history, and potentially pressure history, of the polymer, as well as potentially the pressure at which the measurement is made. The chemical structure of the polymer heavily influences the glass transition by affecting chain mobility.

The “melting temperature,” Tₘ, of a polymer is the temperature at which an endothermal peak is observed in a DSC measurement, and where at least some of the crystallites begin to become disordered. The measured melting temperature may occur over a temperature range as the size of the crystallites, as well as presence of impurities and/or plasticizers, impacts the measured melting temperature of a polymer.

As used herein, a reference to the crystallinity of a polymer refers to the crystallinity as determined by standard DSC techniques.

As used herein, an “active agent” refers to a substance that, when administered in a therapeutically effective amount to a patient suffering from a disease or condition, has a therapeutic beneficial effect on the health and well-being of the patient. A therapeutic beneficial effect on the health and well-being of a patient includes, but it not limited to: (1) curing the disease or condition; (2) slowing the progress of the disease or condition; (3) causing the disease or condition to retrogress; or, (4) alleviating one or more symptoms of the disease or condition.

As used herein, an “active agent” also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease or condition in the first place; (2) maintaining a disease or condition at a retrogressed level once such level has been achieved by a therapeutically effective amount of a substance, which may be the same as or different from the substance used in a prophylactically effective amount; or, (3) preventing or delaying recurrence of the disease or condition after a course of treatment with a therapeutically effective amount of a substance, which may be the same as or different from the substance used in a prophylactically effective amount, has concluded.

As used herein, “active agent” also refers to pharmaceutically acceptable, pharmacologically active derivatives of those active agents specifically mentioned herein, including, but not limited to, salts, esters, amides, and the like.

As used herein, a material that is described as a layer, a film, or a coating “disposed over” an indicated substrate refers to disposition of the material directly or indirectly over at least a portion of the surface of the substrate. “Directly deposited” means that the material is applied directly onto the surface of the substrate. “Indirectly deposited” means that the material is applied to an intervening layer that has been deposited directly or indirectly over the substrate. The terms “layer”, and “coating layer” will be used interchangeably and refer to a layer or film, as described in this paragraph. A coating may comprise one or more layers. A coating on a medical device, such as a stent, does not provide structural support to a lumen. The typical dimensions of a coating are about 50 microns or less than 50 microns, preferably about 20 microns or less than 20 microns, and more preferably about 10 microns or less than 10 microns. A coating may be chemically bonded to the substrate or layer over which it is disposed, but in most embodiments, the coating is not chemically bonded to the substrate or the layer over which it is disposed. One laminate or layer of a device which is a multi-laminate may or may not be a “coating” on a device “body” as defined herein depending upon which layer or layer(s) provide the structural support. If several layers of the laminate are needed to provide structural support, than these layers together constitute the “body,” and one of these layers is not a “coating” as defined herein.

### EXAMPLES

The examples set forth below are for illustrative purposes only and are in no way meant to limit the invention. The following examples are given to aid in understanding the invention, but it is to be understood that the invention is not limited to the particular examples. The parameters and data are not to be construed to limit the scope of the embodiments of the invention.

### Example 1

### Sterilization at 50° C.

Polymeric stents manufactured from poly(L-lactide) (PLLA). Polymer tubes were extruded from Poly(L-lactide) (PLLA), RESOMER® L 210 S, supplied by Boehringer Ingelheim by extrusion of a polymer tube, biaxially expanding the polymer tube such that the radial expansion was about 450% (within about 400% to about 500%) and the axial expansion was about 25% (within about 20% to about 120%), laser cutting a stent pattern, and crimping the stent onto a balloon catheter.

Stents manufactured by the method above were sterilized in an 8 ft³ Research and Development chamber. The standard steps used included those described above for a typical EtO sterilization procedure: pulling a vacuum to remove air; a humidification step and a “humidity dwell” time to obtain a relative humidity in the desired range; EtO injection over a short time period to achieve approximately 400 mg/liter to 600 mg/liter of EtO which is then maintained for a specified time period (roughly about 3 to about 6 hours for this case); and an aeration operation in which the chamber is repeatedly pulsed with steam, nitrogen, air to remove the EtO with an optional final “wash” of air at atmospheric pressure with a potential ambient dwell time to assure removal of all EtO. As noted previously, the chamber pressure set-points for the steam injection and EtO injection to obtain specified humidity and EtO levels are specific for each chamber. In this case, the specified time period for the EtO exposure was longer than if the sterilization had been done at a higher temperature, and this time may vary from the time used in this particular experiment.

The temperature of the chamber during the humidification and other pre-injection steps was maintained between 35° C. and 52° C. During the EtO dwell time, the temperature was set to 40° C., but the actual temperature measured ranged from about 35° C. to about 49° C. Temperature probes indicated that the product temperature reached a temperature of about 50° C., and during most of the EtO dwell time was in the mid 40's ° C. During the EtO dwell time, the humidity ranged from about 66% to about 70% relative humidity.

All of the sterilized stents were tested by expansion on a balloon in water at 37° C. to the nominal diameter (as an example, 3 mm for a 3 mm×18 mm stent), and then inflated under controlled conditions until fracture occurred. During the initial expansion to the nominal diameter, a significant fraction, three of five, of the sterilized stents exhibited one or more broken struts upon deployment to the nominal diameter.

### Example 2

### Sterilization at 30° C./30% RH

Stents manufactured by the method of Example 1 were sterilized in an R & D EtO sterilization chamber set to 30° C. with the humidity control set to 30% relative humidity (RH). The sterilization operations were essentially the same as Example 1 except that the temperature during the EtO dwell time and the duration of the EtO dwell time were changed. The duration of the EtO dwell time increases as the temperature decreases. Measurements made during the EtO dwell time indicated that the temperature was maintained at 32° C. 1° C., and that the humidity, except for one excursion to about 40% RH, was maintained in a range from about 20-25% RH.

The sterilized stents were tested in the same manner described in Example 1. Of the five stents tested, none exhibited one or more broken struts upon deployment to the nominal diameter.

### Example 3

### Sterilization at 30° C./50% RH

Stents manufactured by the method of Example 1 were sterilized in an R & D EtO sterilization chamber set to 30° C. with the humidity control set to 70% RH. The sterilization operations were essentially the same as Example 1 except that the temperature during the EtO dwell time and the duration of the EtO dwell time were changed. Measurements made during the EtO dwell time indicated that the temperature was maintained at 32° C.±1° C., and that the humidity reached about 60% RH maximum briefly and was at about 50% RH for about 3-5 hours.

The sterilized stents were tested in the same manner described in Example 1. Of the five stents tested, none exhibited one or more broken struts upon deployment to the nominal diameter.

While particular embodiments of the present invention have been shown and described, it will be obvious to those skilled in the art that changes and modifications can be made without departing from this invention in its broader aspects. Embodiments of the invention encompass different aspects of the different embodiments that are combined together. Therefore, the appended claims are to encompass within their scope all such changes and modifications as fall within the true spirit and scope of this invention.

What is claimed is:

1. A method comprising:
exposing a medical device, the device comprising a device body comprising a polymer, to a fluid comprising ethylene oxide such that during the exposure the temperature of the device is not greater than 40° C. and not less than 15° C. and such that the sterility assurance level of the device is less than 1×10⁻³ as a result of the exposure to the fluid;
wherein the glass transition temperature of the polymer of the device is between about 35° C. and about 65° C.

2. The method of claim 1, wherein the fluid is a gas, the medical device is an implantable medical device, and the sterility assurance level of the device is less than 1×10⁻⁶ as a result of the exposure to the gas.

3. The method of claim 2, wherein the exposure occurs at a temperature not greater than 35° C.

4. The method of claim 3, wherein the exposure occurs at a temperature not greater than 32° C.

5. The method of claim 2, wherein the gas comprises at least 300 mg/liter ethylene oxide.

6. The method of claim 5, wherein the gas comprises at least 400 mg/liter ethylene oxide.

7. The method of claims 5, wherein the gas comprises not more than 1000 mg/liter ethylene oxide.

8. The method of claim 7, wherein the gas has a humidity level of 20% to 95% relative humidity.

9. The method of claim 8, wherein the gas has a humidity level of not more than 50%.

10. The method of claim 9, wherein the gas has a humidity level of about 30% or less than 30%.

11. The method of claim 2, wherein the device is a stent.

12. The method of claim 11, wherein the stent body comprises at least 80% by volume of the polymer.

13. The method of claim 11, wherein the polymer comprises a continuous phase of the stent body.

14. The method of claim 11, wherein prior to sterilization the stent has an initial modulus, and after sterilization the stent has a modulus of about 70% or greater than 70% of the initial modulus.

15. The method of claim 12, wherein prior to sterilization the stent has an initial modulus, and after sterilization the stent has a modulus of about 80% or greater than 80% of the initial modulus.

16. The method of claim 9, wherein the device is a stent, the stent body substantially consists of poly(L-lactide), and during the exposure the temperature of the device is not greater than 35° C. and not less than 15° C.

17. The method of claim 2, wherein the exposure is from about 1 hour to about 20 hours.

18. The method of claim 17, wherein the exposure is from about 1 hour to about 10 hours.

19. A method comprising:
exposing an implantable medical device, the device comprising a device body, the device body comprising at least 50% by volume of a polymer, to a gas comprising ethylene oxide such that during the exposure the temperature of the device is not greater than 40° C. and not less than 15° C. and such that the sterility assurance level of the device is less than 1×10⁻⁶ as a result of the exposure to the gas;
wherein the polymer is a semi-crystalline biodegradable polymer.

20. The method of claim 19, wherein the biodegradable polymer is poly(L-lactide), polymandelide, poly(DL-lactide), polyglycolide, poly(L-lactide-co-glycolide), or any block, alternating, or random copolymer thereof, or any block, alternating, or random copolymer of at least one of the group of consisting of poly(L-lactide), polymandelide, poly(DL-lactide), polyglycolide, and poly(L-lactide-co-glycolide), and at least one of the group of consisting of polycaprolactone, poly(trimethylene carbonate), polydioxanone, poly(4-hydroxy butyrate), and poly(butylene succinate), or any blend thereof.

21. A method of fabricating a polymeric stent comprising:
forming a tube, the tube comprising a polymer;
cutting a stent pattern into the tube to form a polymeric stent; and
exposing the polymeric stent to a gas comprising ethylene oxide such that the temperature of the stent is not greater than 40° C. and not less than 15° C. during the exposure, and such that the sterility assurance level of the device is about 1×10⁻⁶ or less than 1×10⁻⁶ as a result of the exposure to the gas;
wherein the glass transition temperature of the polymer of the device is between about 35° C. and about 65° C.

22. The method of claim 21, wherein the polymer is poly(L-lactide), poly(D,L-lactide), polyglycolide, poly(L-lactide-co-glycolide), or any blend thereof.

23. The method of claim 21, the method further comprising radially expanding the polymeric tube prior to cutting a stent pattern in the tube, the expansion occurring at a temperature from about T_{g} to about Tₘ of the polymer of the polymeric tube which increases radial strength and the crystallinity of the polymeric tube.

24. The method of claim 23, wherein the tube comprises poly(L-lactide), the poly(L-lactide) comprising at least a continuous phase of the tube, the exposure occurs at a temperature of about 32° C. or less than 32° C., and the coating comprises poly(D,L-lactide) and a active agent selected from the group consisting of everolimus, sirolimus, biolimus, paclitaxel, zotarolimus, and combinations thereof.

25. A method comprising:
exposing an implantable medical device, the device comprising a device body comprising a polymer, to a fluid comprising ethylene oxide and between about 20% and 50% relative humidity such that during the exposure the temperature of the device is not greater than the wet glass transition temperature of the polymer, and such that the sterility assurance level of the device is less than 1×10⁻⁴ as a result of the exposure to the gas.

26. The method of claim 25, wherein the glass transition temperature of the polymer of the device is between about 40° C. and about 65° C.

27. The method of claim 26, wherein the glass transition temperature of the polymer of the device is between about 50° C. and about 60° C.

28. The method of claim 27, wherein during the exposure the temperature of the device is not greater than 40° C. and not less than 15° C.

29. The method of claim 25, wherein the sterility assurance level of the device is less than 1×10⁻⁶ as a result of the exposure to the gas.
